(19) United States
(12) Patent Application Publication
(10) Pub. No.: US 2024/0197008 A1
(43) Pub. Date: Jun. 20, 2024

(54) MULTILAYER PROTECTIVE ANTIMICROBIAL MASK COMPRISING NANOFIBER MEMBRANE

(71) Applicant: ARGAMAN TECHNOLOGIES LTD., Migdal Haemek (IL)

(72) Inventor: Jeffrey Gabbay, Jerusalem (IL)

(21) Appl. No.: 17/909,829

(22) PCT Filed: Mar. 10, 2021
(86) PCT No.: PCT/IL2021/050264

§ 371 Date: Sep. 7, 2022

Related U.S. Application Data
(60) Provisional application No. 62/987,360, filed on Mar. 10, 2020.

(51) Int. Cl. A41D 13/11; A01N 25/10; A01N 59/16; A01N 59/20; A01P 1/00; B32B 5/02; B32B 5/06; B32B 5/26; D01F 1/10; D01F 6/62; D03D 15/217; D03D 15/283; D03D 15/50; D04B 1/14; D04B 1/24; D04B 21/14; D04B 21/20; D04H 1/413; D04H 1/4258; D04H 1/435; D04H 1/4382; D04H 1/498; D04H 1/56; D04H 1/728; D06M 10/06; D06M 11/42; D06M 16/00; D06M 101/06

(52) U.S. Cl. CPC A41D 13/1192; A01N 25/10; A01N 59/16; A01N 59/20; A01P 1/00; B32B 5/022; B32B 5/026; B32B 5/06; B32B 5/266; B32B 5/279; D01F 1/103; D01F 6/62; D03D 15/217; D03D 15/283; D03D 15/50; D04B 1/14; D04B 1/24; D04B 21/14; D04B 21/207; D04H 1/413; D04H 1/4258; D04H 1/435; D04H 1/43835; D04H 1/498; D04H 1/56; D04H 1/728; D06M 10/06; D06M 11/42; D06M 16/00; A41D 2500/10; A41D 2500/20; A41D 2500/30; B32B 2250/20; B32B 2250/40; B32B 2262/0223; B32B 2262/0276; B32B 2262/04; B32B 2262/062; B32B 2262/144; B32B 2264/102; B32B 2307/72; B32B 2307/7376; B32B 2571/00; D06M 2101/06; D10B 2201/02; D10B 2331/04; D10B 2401/13; D10B 2501/04

(57) ABSTRACT

The present invention relates to a multilayer protective mask having antimicrobial properties, said mask comprising a body portion comprising: at least a first and a second fabric layers having random fiber configuration; a middle layer comprising a nanofiber membrane; and a third and a fourth fabric layers. The first fabric layer is disposed between the third fabric layer and the nanofiber membrane, and the second fabric layer is disposed between the fourth fabric layer and the nanofiber membrane. The first, the second, the third, and the fourth fabric layers have incorporated therein a synergistic combination of at least two metal oxide powders, comprising a mixed oxidation state oxide of a first metal and a single oxidation state oxide of a second metal, said combination having synergistic antimicrobial properties.

### FIELD OF THE INVENTION

The present invention relates to multilayer safety masks having antimicrobial activity comprising fabric layers incorporating a biocidal combination of metal oxides, and a nanofiber membrane.

### BACKGROUND OF THE INVENTION

Respirators and surgical masks (face masks) are examples of personal protective equipment that are used to protect the wearer from airborne particles and from liquid contaminating the face. The use of face masks is a recommended practice in the healthcare industry to help prevent the spread of disease. Face masks worn by healthcare providers help reduce infections in patients by filtering the air exhaled from the wearer, thus reducing the number of harmful organisms or other contaminants released into the environment. Additionally, face masks protect healthcare workers by filtering airborne contaminants and microorganisms from the inhaled air.

One of the major problems of surgical masks and respirators is that they cannot be worn for more than a limited period of time before the holes in the filtration strata thereof get clogged by the wearer's nasal and mouth vapor, which clogs the holes in the filtration level making respiration difficult or impossible. Using materials with larger pores is, however, disadvantageous, as it would reduce the protective efficiency of the mask. Accordingly, the textile used in the preparation of the filter masks has to provide high breathability without compromising the filtering efficiency thereof.

Protective masks can be made of a woven or a non-woven fabric. In general, surgical masks are produced from a woven fabric, which can be configured for a multiple use, while respirators are generally produced from a non-woven material and are intended for single use.

Nanofibers, which are commonly employed in filtering systems, have recently been reported as being suitable for use in face masks and respirators (Akduman, Cigdem & Akumbasar, Perrin. (2018), Nanofibers in face masks and respirators to provide better protection. IOP Conference Series: Materials Science and Engineering, 460, 012013).

The recent outbreak of Coronavirus Disease 2019 (COVID-19) caused by the SARS-COV-2 virus has elevated interest in antibacterial masks which can deactivate microbes and viruses in the vicinity of the face mask, so that they are not inhaled by a wearer or transferred to another surface by inadvertent contact of the mask with other surfaces or user's hands.

U.S. Pat. No. 7,845,351 is directed to a face mask for reducing the amount of microbes to which a wearer is exposed. The face mask includes a body portion that has an outer layer that has been treated with a germicidal agent in an effective amount. The layer may be a nonwoven fabric like a spunbond, meltblown or coform layer and may be a laminate of such layers. The face mask having such a germicidal treatment can result in a reduction in microbial activity as compared to another face mask, identical but for the germicidal agent.

U.S. Pat. No. 7,700,501 is directed to an adsorptive filtering material with biological and chemical protective function, in particular with protective function with regard to both chemical and biological poisons and noxiants, such as chemical and biological warfare agents, the adsorptive filtering material having a multilayered construction comprising a first outer supporting layer and a second outer supporting layer and an adsorptive layer disposed between the two supporting layers, the adsorptive filtering material further comprising at least one catalytically active component, the first outer supporting layer and/or the second outer supporting layer being provided with the catalytically active component.

WO 2009/146412 is directed to a facial mask for decreasing the transmission of one or more than one human pathogen to and from a human wearer of the facial mask.

WO 2016/125173 is directed to antimicrobial fabric materials suitable for use in protective masks, first responder protective clothing and hospital garments, said materials comprising a polymer having incorporated therein a synergistic combination of at least two metal oxide powders, comprising a mixed oxidation state oxide of a first metal and a single oxidation state oxide of a second metal. A protective mask disclosed therein comprises a fabric material having a maximal thickness of 3.2 mm, surface density of from about 5 to about 30 g/m², and an air permeability of from about 150 to about 6000 L/m³.

It is known that certain individual metal oxides, when exposed to moisture, will release ions to the environment in which the metal oxide is exposed. It is also known that these ions have antimicrobial, antiviral, and anti-fungal properties (Borkow and Gabbay, FASEB J. 2004 Nov. 18 (14):1728-30), as well as anti-mite qualities (Mumchuoglu, Gabbay, Borkow, International Journal of Pest Management, Vol. 54, No. 3, 2008, 235-240).

U.S. Pat. No. 6,482,424 discloses a method for combating and preventing nosocomial infections, comprising providing to health care facilities textile fabrics incorporating fibers coated with an oxidant, cationic form of copper, for use in patient contact and care, wherein the textile fabric is effective for the inactivation of antibiotic resistant strains of bacteria.

U.S. Pat. No. 6,436,420 is related to fibrous textile articles possessing enhanced antimicrobial properties prepared by the deposition or interstitial precipitation of tetrasilver tetroxide (Ag₄O₄) crystals within the interstices of fibers, yarns and/or fabrics forming such articles.

There remains an unmet need for a cost-effective safety mask having antimicrobial and antiviral properties and enhanced air permeability, which would improve user compliance and protection efficiency of the mask, in particular against airborne viruses.

### SUMMARY OF THE INVENTION

The present invention relates to safety masks which have antimicrobial properties. The mask contains multiple textile layers, which are treated with an antimicrobial composition. The middle layer of the safety mask of the present invention includes a nanofiber membrane, which affords for the low thickness and high air permeability of the mask, while maintaining the intrinsic antimicrobial properties and high filtering efficiency intact. Advantageously, the nanofiber membrane does not have to be treated with the antimicrobial composition in order for the mask to have the desired germicidal and antiviral properties. The particular combination of the textile layers having incorporated therein a synergistic antimicrobial composition comprising at least two metal oxides, and the nanofiber membrane, provides the low-cost antibacterial safety mask of the present invention, which protection factor is as high as that of commercial ASTM 2100 Level 2 respirators, and which germicidal efficiency and breathability are significantly higher.

During the Covid 19 pandemic it has become particularly important that face masks protect both the mask wearer and people around him. The present invention is based in part on the surprising finding that in order to deactivate a virus in less time than it takes for the virus to penetrate the mask, five layers are required, wherein four layers are composed of a fabric having biocidal properties, said four layers being designed to both deactivate the virus and delay its passage, and a middle layer, being a nanofiber membrane that significantly diminishes the passage of the virus therethrough. In order to enhance protection of both the wearer from an airborne virus and people in his close vicinity, in case the wearer is infected (i.e., to prevent transmission of the virus from outside the mask, as well as its transmission from the inner layers outwards), it is particular important that as much as 99.9% of the virus is deactivated before it reaches the middle layer, which is the nanofiber membrane.

It has been shown by the inventors of the present invention that each individual fabric layer deactivates between 95% and 99.9% of the SARS-COV-2 virus within 5 minutes. The nanofiber membrane, being the middle layer, was found to have a viral filtration efficiency of about 99.8%. The nanofiber membrane therefore acts to delay the passage of the virus and holds it in place so that the biologically active fabric layers can continue deactivating the virus to almost completely prevent its passage to the opposite side of the membrane. Even if a minute amount of the virus passes two fabric layers with the 95% and 99.9% virus deactivation rate and the nanofiber membrane, it is deactivated by the two additional fabric layers on the opposite side of the membrane, also having the 95% and 99.9% virus deactivation rate. Without wishing to being bound by theory or mechanism of action, it is contemplated that the particular combination of the 4 biocidal fabric layers and the nanofiber membrane therefore makes the chances of a virus passing from one side of the mask to another statistically impossible.

In one aspect, there is provided a multilayer safety mask having antimicrobial properties, said mask comprising a body portion comprising a first and a second fabric layers having random fiber configuration; a middle layer comprising a nanofiber membrane; and a third and a fourth fabric layers, wherein the first fabric layer is disposed between the third fabric layer and the nanofiber membrane and wherein the second fabric layer is disposed between the fourth fabric layer and the nanofiber membrane, and wherein the first, the second, the third, and the fourth fabric layers have incorporated therein a synergistic combination of at least two metal oxide powders, comprising a mixed oxidation state oxide of a first metal and a single oxidation state oxide of a second metal, said combination having synergistic antimicrobial properties, wherein the ions of the metal oxides are in ionic contact upon exposure of said fabric layers to moisture.

According to some embodiments, the first and the second fabric layers are in a form of a needle punch fabric.

According to some embodiments, the third and the fourth fabric layers are in a form of a non-woven fabric, selected from the group consisting of a spun bond fabric, melt blown fabric and combinations thereof. According to certain embodiments, the third and the fourth fabric layers are in a form of a spun bond fabric.

According to some embodiments, the first, the second, the third, and the fourth fabric layers have a thickness of from about 10 μm to about 1000 μm. In further embodiments, first, the second, the third, and the fourth fabric layers have a surface density of from about 5 g/m² to about 70 g/m². In still further embodiments, the first, the second, the third, and the fourth fabric layers have a mean pore size of at least about 30 μm.

The third and the fourth fabric layers can be in a form of a woven or knit fabric. In some embodiments, the woven or knit fabric has a surface density of from about 5 g/m² to about 70 g/m² and a mean pore size of from about 20 μm to about 60 μm.

The first, the second, the third, and the fourth fabric layers can be made of a synthetic material, semi-synthetic material, natural material, or any combination thereof. In some embodiments, the synthetic material or semi-synthetic material comprises a polymer selected from the group consisting of polyalkene, polyester, polyamide, polyaramid, cellulose-based polymer, and combinations thereof. The polyalkene can be selected from the group consisting of polypropylene, polyethylene and combinations thereof. In some embodiments, the natural material is selected from the group consisting of cotton, silk, wool, linen and combinations thereof.

In certain embodiments, the first and the second fabric layers are made of a combination of cotton, viscose, and polyester. In further embodiments, the first and the second fabric layers are in a form of a needle punch fabric.

In certain embodiments, the third and the fourth fabric layers are made of polypropylene. In further embodiments, the third and the fourth fabric layers are in a form of a spun bond fabric.

In certain embodiments, the third and the fourth fabric layers are made of cotton. In further embodiments, the third and the fourth fabric layers are in a form of a woven fabric.

In certain embodiments, the third and the fourth fabric layers are made of a combination of cotton and polyester. In further embodiments, the third and the fourth fabric layers are in a form of a woven fabric.

According to some embodiments, the combined weight of the at least two metal oxide powders within the fabric layers constitutes from about 0.05% (w/w) to about 5% (w/w) of the total weight of each one of the first fabric layer, of the second fabric layer, of the third fabric layer, and of the fourth fabric layer.

According to some embodiments, the mixed oxidation state oxide is selected from the group consisting of tetrasilver tetroxide (Ag₄O₄), Ag₃O₄, Ag₂O₂, tetracopper tetroxide (Cu₄O₄), Cu (I, III) oxide, Cu (II, III) oxide and combinations thereof. According to some embodiments, the single oxidation state oxide is selected from the group consisting of copper oxide, silver oxide, zinc oxide and combinations thereof. In certain embodiments, the synergistic combination of the at least two metal oxide powders comprises copper oxide and tetrasilver tetroxide.

According to some embodiments, the mixed oxidation state oxide constitutes up to about 15% (w/w) of the total weight of the synergistic combination of the at least two metal oxide powders. According to further embodiments, the mixed oxidation state oxide is present in the synergistic combination in a detectable amount.

According to some embodiments, each one of the first fabric layer and the second fabric layer constitutes between about 15% and 45% of the total weight of the body portion of the mask.

According to some embodiments, each one of the third fabric layer and the fourth fabric layer constitutes between about 15% and 45% of the total weight of the body portion of the mask.

The nanofiber membrane of the safety mask can be electrospun or melt blown. In some embodiments, the nanofiber membrane has a thickness of from about 1 μm to about 100 μm. In certain embodiments, the nanofiber membrane has a thickness of from about 5 μm to about 20 μm.

According to some embodiments, the nanofiber membrane has a surface density of from about 0.5 g/m² to about 10 g/m². According to further embodiments, the nanofiber membrane has a mean pore size of from about 40 nm to about 100 nm. In yet further embodiments, the nanofiber membrane has a porosity of from about 70% to about 90%. In still further embodiments, the nanofiber membrane has a mean fiber diameter of from about 100 nm to about 800 nm.

The nanofiber membrane can be made of a natural, synthetic or semi-synthetic polymer material selected from the group consisting of polyvinyl alcohol (PVA), polyvinyl chloride (PVC), cellulose, polyethylene, polysulfone, Nylon, polyacrylonitrile, polystyrene, polyethylene oxide (PEO), polyethylene-terephthalate (PET), polyethylene-naphthalate (PEN), and combinations thereof. In certain embodiments, the nanofiber membrane is made of PVA.

According to some embodiments, the nanofiber membrane constitutes between about 2% and 10% of the total weight of the body portion of the mask.

According to some embodiments, the body portion is configured to be placed over a mouth and at least part of a nose of a user such that respiration air is drawn through said body portion. According to further embodiments, the mask further comprises a fastening member, which is connected to the body portion of the mask and configured for attaching the body portion to the user.

The mask can be in a form of a fold-flat surgical mask or a molded cup-shaped mask.

In some embodiments, the mask has air permeability of at least about 70% air flow.

In some embodiments, the mask is a single-use mask, wherein the third and the fourth fabric layers are in a form of a spun bond fabric, which is made of polypropylene. In some embodiments, the mask is a multiple-use mask, wherein the third and the fourth fabric layers are in a form of a woven fabric, which is made of cotton, polyester or any combination thereof. The synergistic combination of at least two metal oxide powders can be incorporated into cotton fibers, polyester fibers, or both, of the third and the fourth fabric layers. In some related embodiments, the first and the second fabric layers are in a form of a needle punch fabric, which is made of a combination of cotton, polyester, and viscose. According to some embodiments, the synergistic combination of at least two metal oxide powders is incorporated into polyester fibers, cotton fibers, or both, of the first and the second fabric layers.

In some embodiments, the mask is for use in combating or inhibiting the activity of microbes or micro-organisms, selected from the group consisting of gram-positive bacteria, gram-negative bacteria, fungi and viruses. In certain embodiments, the virus is an airborne virus. The virus can be selected from the group consisting of a coronavirus, a rhinovirus, an influenza A and/or B virus, a parainfluenza 1, 2 and/or 3 virus, and any combination thereof. In some currently preferred embodiments, the coronavirus is SARS-COV-2.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

Some embodiments of the invention are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the invention. For the sake of clarity, some objects depicted in the figures are not to scale.

FIG. 1A: Schematic representation of the single-use mask layers comprising third and fourth fabric layers, which are woven fabrics, in accordance with some embodiments of the invention.

FIG. 1B: Schematic representation of the single-use mask layers comprising third and fourth fabric layers, which are non-woven fabrics, in accordance with some embodiments of the invention.

FIG. 1C: Schematic representation of the multiple-use mask layers, in accordance with some embodiments of the invention.

### DETAILED DESCRIPTION

The present invention relates to safety masks, which afford high breathability without compromising protection to the wearer, and particularly protection related to viral deactivation. The multilayer mask comprises a combination of antimicrobial fabric layers, which are configured to destroy the viruses and microbes, which are present on the surface of the fabric and to deactivate the viruses and microbes, which pass through the fabric in the uniquely designed use of an air permeable mask filtering system. The antimicrobial fabric layers contain a synergistic antibacterial combination of at least two metal oxide powders homogeneously incorporated therein. The improved antimicrobial activity of the mask of the present invention is achieved without increasing the thickness and/or density of the filtering layers. Instead, a very thin nanoporous nanofiber membrane is disposed between the antimicrobial fabric layers, which effectively prevents the passage of various airborne particles, including viruses and bacteria, without requiring additional filter layers.

In one aspect, there is provided a multilayer safety mask having antimicrobial properties, said mask comprising a body portion comprising a first and a second fabric layers having random fiber configuration; a middle layer comprising a nanofiber membrane; and a third and a fourth fabric layers, wherein the first fabric layer is disposed between the third fabric layer and the nanofiber membrane and wherein the second fabric layer is disposed between the fourth fabric layer and the nanofiber membrane, and wherein the first, the second, the third, and the fourth fabric layers have incorporated therein a synergistic combination of at least two metal oxide powders, comprising a mixed oxidation state oxide of a first metal and a single oxidation state oxide of a second metal, said combination having synergistic antimicrobial properties, wherein the ions of the metal oxides are in ionic contact upon exposure of said fabric layers to moisture.

In another aspect, there is provided method of preventing transmission of viral pathogens between a source of viral pathogens and a target of said viral pathogens comprising positioning between said source and said target a multilayer safety mask having antimicrobial properties, said mask comprising a body portion comprising a first and a second fabric layers having random fiber configuration; a middle layer comprising a nanofiber membrane; and a third and a fourth fabric layers, wherein the first fabric layer is disposed between the third fabric layer and the nanofiber membrane and wherein the second fabric layer is disposed between the fourth fabric layer and the nanofiber membrane, and wherein the first, the second, the third, and the fourth fabric layers have incorporated therein a synergistic combination of at least two metal oxide powders, comprising a mixed oxidation state oxide of a first metal and a single oxidation state oxide of a second metal, said combination having synergistic antimicrobial properties, wherein the ions of the metal oxides are in ionic contact upon exposure of said fabric layers to moisture.

Fabric material consists of a network of fibers which can be aligned or dispersed in a woven or non-woven fashion.

The term “non-woven fabric” is meant to encompass fabrics which are neither woven nor knitted.

The terms “fabric” and “textile” are used herein interchangeably.

A non-woven fabric is a fabric-like material made from stable or filament fibers, bonded together by chemical, mechanical, heat or solvent treatment. Non-woven fabrics are often classified according to the procedures used for their preparation, including, among others, water thorn non-woven fabric, thermal bonding non-woven fabric, pulp flow into nets non-woven fabric, wet non-woven fabric, spinning sticky non-woven fabric, weld spray non-woven fabric, needle punch non-woven fabric, and sewing make up non-woven fabric.

Non-woven fabrics comprising staple fibers are typically made in 4 steps. Fibers are first spun, cut to a few centimeters' length, and put into bales. The staple fibers are then blended, “opened” in a multistep process, dispersed on a conveyor belt, and spread in a uniform web by a wetlaid, airlaid, or carding/crosslapping process. Staple non-woven fabrics can be bonded either thermally or by using resin. Bonding can be throughout the web by resin saturation or overall thermal bonding or in a distinct pattern via resin printing or thermal spot bonding.

Needle punch is a process for making a nonwoven textile in which a continuous mat of randomly laid fibers or filaments is entangled with barbed needles, thereby binding the fibers together.

Spun Bond (SB) is a process for producing fibrous webs or articles directly from polymers or resins using high-velocity air or another appropriate force to attenuate filament fibers. The SB process can be used to produce nano- or micro-fibers. SB fibers generally have thickness in the range of 2 to 4 μm, although they may be as small as 0.1 μm and as large as 10 to 15 μm.

Melt-blown non-woven fabrics are typically produced by extruding melted polymer fibers through a spin net or die consisting of up to 40 holes per inch to form long thin fibers which are stretched and cooled by passing hot air over the fibers as they fall from the die. The resultant web can be collected into rolls and subsequently converted to finished products.

According to some embodiments, the first and/or the second fabric layers comprise non-woven fabrics.

According to some embodiments, the third and/or the fourth fabric layers comprise non-woven fabrics.

According to some embodiments, the first and the second fabric layers are in a form of a needle punch fabric.

According to some embodiments, the third and the fourth fabric layers are in a form of a spun-bond material, a melt-blown material or a combination thereof. According to some embodiments, the third and the fourth fabric layers are in a form of a spun-bond material.

The fabric suitable for use in the safety masks according to the principles of the present invention can have a surface density of from about 1 g/m² to about 500 g/m². In some embodiments, the fabric has a surface density of from about 2 g/m² to about 200 g/m², of from about 5 g/m² to about 100 g/m², of from about 5 g/m² to about 50 g/m², of from about 5 g/m² to about 30 g/m², of from about 7 g/m² to about 25 g/m², or of from about 10 g/m² to about 20 g/m². Each possibility represents a separate embodiment of the invention.

The fabric suitable for use in the first, second, third, and/or fourth fabric layers of the safety masks according to the principles of the present invention can have a thickness of from about 10 μm to about 1000 μm. In some embodiments, the fabric suitable for use in the first, second, third, and/or fourth fabric layers of the safety masks has a thickness of from about 100 μm to about 1000 μm. In further embodiments, the first, the second, the third, and/or the fourth fabric layers have a thickness of from about 100 μm to about 750 μm.

According to some embodiments, the fabric has a mean pore size of from about 5 μm to about 100 μm. In further embodiments, the fabric has a mean pore size of from about 15 μm to about 100 μm. Each possibility represents a separate embodiment of the invention. According to some embodiments, the pores are formed between the fibers of the woven or non-woven fabric.

The fabric suitable for use in the safety masks according to the principles of the present invention can have an air-permeability of from about 50 L/m³ to about 10000 L/m³. In further embodiments, the fabric has an air-permeability of from about 100 L/m³ to about 8000 L/m³, of from about 150 L/m³ to about 6000 L/m³, of from about 200 L/m³ to about 5000 L/m³, or of from about 500 L/m³ to about 2500 L/m³. Each possibility represents a separate embodiment of the invention.

In some embodiments, the fabric comprises staple fibers or filament fibers. A staple fiber is a fiber of a standardized length, which can be twisted into a yarn. A filament fiber is a fiber that comes in continuous to near continuous lengths. Synthetic fibers can be manufactured as stable or filament fibers. If the filament fiber is cut into discrete lengths, it becomes staple fiber. According to some embodiments, the fiber comprises the at least two metal oxide powders incorporated therein.

The synthetic fiber can be obtained by an extrusion, molding, casting or 3D printing process. In certain embodiments, the fiber is an extruded fiber.

In some embodiments, the fiber of the first, the second, the third and the fourth fabric layers have a micrometric thickness. Micrometric fibers can be produced, for example, by spinning. In some embodiments, the fiber has a thickness of from about 1 μm to about 150 μm. In further embodiments, the fiber has a thickness of from about 10 μm to about 100 μm. The term “thickness”, as used herein, refers to a size of the fiber in the shortest dimension thereof. If the fiber has a circular or a circular-like cross section, the thickness refers to a diameter of the fiber.

Woven fabrics are produced by the interlacing of warp (0°) fibers and weft (90°) fibers in a regular pattern or weave style. The integrity of the fabric is maintained by the mechanical interlocking of the fibers. Weave style, which defines various fabric characteristics can include, inter alia, plain, twill, satin, basket, leno and mock leno.

According to other embodiments, the third and/or the fourth fabric layers are in a form of a woven fabric.

In some embodiments, the woven fabric includes a knitted fabric. In further embodiments. the third and/or the fourth fabric layers are in a form of a knitted fabric.

According to some embodiments, the woven fabric of the third fabric layer and/or of the fourth fabric layer has a surface density of from about 1 g/m² to about 70 g/m². In further embodiments, the woven fabric has a surface density of from about 2 g/m² to about 60 g/m², of from 2 g/m² to about 50 g/m². of from 2 g/m² to about 40 g/m², of from about 5 g/m² to about 30 g/m², of from about 7 g/m² to about 25 g/m², or of from about 10 g/m² to about 20 g/m². Each possibility represents a separate embodiment of the invention.

According to some embodiments, the woven fabric of the third fabric layer and/or of the fourth fabric layer has a thickness of from about 2 μm to about 80 μm. The thickness can be increased by layering of multiple sheaths.

According to some embodiments, the woven fabric of the third fabric layer and/or of the fourth fabric layer comprises staple fibers. In some embodiments, the fiber of the woven fabric has a thickness of from about 50 nm to about 150 μm. In further embodiments, the fiber has a thickness of from about 100 nm to about 100 μm, of from about 200 nm to about 50 μm, or from about 500 nm to about 10 μm.

According to some embodiments, the non-woven fabric, the woven fabric or both comprise a polymer. The synergistic combination of the metal oxides can be incorporated into said polymer, e.g., into the fibers made of said polymer. As used herein, the term “polymer” or “polymeric” refers to materials consisting of repeated building blocks called monomers. The polymer may be homogenous or heterogeneous in its form; hydrophilic or hydrophobic; natural, synthetic, mixed synthetic or bioplastic. The non-limiting examples of polymers suitable for incorporation of the metal oxide powders include, inter alia, polyalkene, polyester, polyaramid, cellulose-based polymer or a mixture of different cellulose materials, converted cellulose mixed with plasticizers such as but not limited to rayon viscose, starch-based polymer, and acetate; and combinations thereof. Each possibility represents a separate embodiment of the invention.

According to some embodiments, the polymer is a synthetic polymer, including organic polymers, inorganic polymers and bioplastics. According to some embodiments, the polymer is selected from the group consisting of polyalkene, polyester, polyamide, polyaramid, cellulose-based polymers, starch-based polymer, derivatives, dispersions and combinations thereof. Each possibility represents a separate embodiment of the invention. Non-limiting examples of polyalkene include polypropylene and polyethylene. Non-limiting examples of the cellulose-based polymer are viscose or rayon. Non-limiting examples of the polyester include polylactic acid (PLA), polyglycolic acid (PGA), and poly(lactic-co-glycolic acid) (PLGA). The polymer may be water based or solvent based. Combinations of more than one of said materials can also be used provided they are compatible or adjusted for compatibility.

According to certain embodiments, the polymer is selected from the group consisting of polyalkene, polyester, cellulose-based polymers and combinations thereof. According to particular embodiments, the polymer is selected from polypropylene, polyethylene, PLA, PGA, PLGA, rayon, viscose and combinations thereof. In further embodiments, the polymer is selected from polypropylene and polyethylene. In some exemplary embodiments, the polymer is polypropylene.

According to some embodiments, the non-woven fabric of the first, second, third and/or fourth fabric layers comprises a polymer selected from the group consisting of polyalkene, polyester, polyamide, polyaramid, cellulose-based polymers, starch-based polymer, derivatives, dispersions and combinations thereof. Each possibility represents a separate embodiment of the invention. In certain embodiments, the fibers of the non-woven fabric are made of said polymer.

In one embodiment, the polymer comprises polyester. In one embodiment, the polymer comprises a polyalkene, preferably a polypropylene. In additional embodiments, the polymer comprises viscose.

According to some embodiments, the woven fabric of the third fabric layer and/or of the fourth fabric layer comprises a polymer selected from the group consisting of polyalkene, polyester, polyamide, polyaramid, cellulose-based polymers, starch-based polymer, derivatives, dispersions and combinations thereof. Each possibility represents a separate embodiment of the invention. In certain embodiments, the fibers of the woven fabric are made of said polymer. In one embodiment, the polymer comprises polyester.

According to some embodiments, the non-woven fabric, the woven fabric or both comprise a natural material. The natural material may be selected from the group consisting of cotton, silk, wool, linen and combinations thereof. In some exemplary embodiments, said natural material is cotton. The synergistic combination of the metal oxides can be incorporated into said natural material.

According to some embodiments, the non-woven fabric, the woven fabric or both comprise a polymer and a natural material. In further embodiments, the polymer is blended with the natural material, e.g., a polymer fiber is blended with a natural fiber. The synergistic combination of the metal oxides can be incorporated into the polymer, which is blended with the natural material. Additionally or alternatively, the synergistic combination of the metal oxides can be incorporated into the natural material, which is blended with the polymer. In some embodiments, the synergistic combination of the metal oxides is incorporated into the natural material and into the polymer.

In some embodiments, the first, the second, the third, and/or the fourth fabric layers comprise cotton, which contains the synergistic combination of metal oxides. In certain embodiments, the third, and/or the fourth fabric layers consist essentially of cotton, which contains the synergistic combination of metal oxides. In other embodiments, said cotton is mixed with polymeric fibers. The cotton can be further mixed with viscose.

In additional embodiments, the first, the second, the third, and/or the fourth fabric layers comprise a modified cellulose fiber. The non-limiting examples of cellulose modified fiber include viscose and rayon.

According to some embodiments, the natural material is present in the first, the second, the third, and/or the fourth fabric layers in a weight percent of up to about 95% of the total weight of the fabric layer. In further embodiments, the first and/or the second fabric layers comprise from about 5% to about 50% wt. of the natural material. In yet further embodiments, the first and/or the second fabric layers comprise from about 10% to about 30% wt. of the natural material. In certain embodiments, the first and/or the second fabric layers comprise about 20% wt. of the natural material. In additional embodiments, the third and/or the fourth fabric layers comprise from about 25% to about 75% wt. of the natural material. In further embodiments, the third and/or the fourth fabric layers comprise from about 40% to about 60% wt. of the natural material. In certain embodiments, the third and/or the fourth fabric layers comprise about 50% wt. natural material. In some related embodiments, the natural material is cotton.

According to some embodiments, the polymer is present in the first, the second, the third, and/or the fourth fabric layers in a weight percent of up to about 95% of the total weight of the fabric layer. In further embodiments, the first and/or the second fabric layers comprise from about 65% to about 95% wt. of the polymer. In yet further embodiments, the first and/or the second fabric layers comprise from about 70% to about 90% wt. of the polymer. In certain embodiments, the first and/or the second fabric layers comprise about 80% wt. of the polymer. In additional embodiments, the third and/or the fourth fabric layers comprise from about 25% to about 75% wt. of the polymer. In further embodiments, the third and/or the fourth fabric layers comprise from about 40% to about 60% wt. of the polymer. In certain embodiments, the third and/or the fourth fabric layers comprise about 50% wt. polymer. In some related embodiments, the polymer is selected from polyester, viscose, and polypropylene.

The synergistic combination of the at least two metal oxide powders comprises a mixed oxidation state oxide of a first metal and a single oxidation state oxide of a second metal, wherein the ions of the metal oxides are in an ionic contact upon hydration of the fabric layers or its exposure to residual moisture. Said synergistic combination has antimicrobial properties.

As used herein, the term “ionic contact” refers to the ability of ions of each of the metal oxide powders, being incorporated within the fabric, to flow to a mutual moisture reservoir, upon exposure to said reservoir. Without wishing to being bound by theory or mechanism of action, it is contemplated that the moisture present in the air or the breath and/or drops of nasal or throat secretion of the wearer of the mask is sufficient to allow the ionic contact between the ions of the at least two metal oxide powders.

As used herein, the term “antimicrobial” refers to an inhibiting, microcidal or oligodynamic effect against microbes, pathogens, and microorganisms, including but not limited to enveloped viruses, non-enveloped viruses, gram-positive bacteria, gram-negative bacteria, fungi, parasites, mold, yeasts, spores, algae, protozoa, acarii and dust mites, amongst others, and subsequent anti-odor properties. In some currently preferred embodiments, the synergistic combination of metal oxides has antiviral properties. In certain embodiments, the virus is an airborne virus.

In order to improve antimicrobial properties of a single oxidation state metal oxide, a mixed oxidation state metal oxide compound is added to the single oxidation state oxide. Without wishing to being bound by theory or mechanism of action, in order to provide the induced biocidal activity, the metal oxide particles should be mixed together in such a manner that the particles of each oxide are exposed to the same moisture reservoir, thus enabling a diffusion of ions from each metal oxide compound to the mutual moisture reservoir. According to some currently preferred embodiments, the mixed oxidation state oxide of a first metal and the single oxidation state oxide of a second metal are present in a uniform mixture within the first, the second, the third, and the fourth fabric layers.

The synergistic combination of the two metal oxides, wherein at least one of the metal oxides is a mixed oxidation state oxide and at least one of the metal oxides is a single oxidation state oxide is a non-naturally occurring biologically active combination. According to some embodiments, said non-naturally occurring combination of metal oxides applied to a fabric demonstrates greater ionic activity than the naturally occurring compounds alone. Without wishing to being bound by theory or mechanism of action, the increased ionic activity is responsible for a greater biocidal effect when compared to the equal amounts of naturally occurring metal oxide compounds under similar conditions.

As defined herein, the term “synergistic combination” refers to a combination of at least two metal oxides, which provides higher antimicrobial efficiency than the equal amount of each of the metal oxides alone. The higher antimicrobial efficiency may relate to accelerated bacteria or micro-organism killing rate.

The synergistic combination applied to the fabric layers comprises two or more biologically active relatively insoluble metal oxides, wherein at least one metal oxide is selected from single oxidation state oxide compounds, and at least one metal oxide is selected from mixed oxidation state oxide compounds has been found to be biologically active by itself and synergistic, providing accelerated virus and microbe mortality as compared to the same single and mixed oxidation state metal oxides individually.

As used herein, the term “mixed oxidation state” refers to atoms, ions or molecules in which the electrons are to some extent delocalized via various electronic transition mechanisms and are shared amongst the atoms, creating a conjugated bond which affects the physiochemical properties of the material. In the mixed oxidation state, electronic transitions form a superposition between two single oxidation states. This can be expressed as any metal that has more than a single oxidation state coexisting, as in the formula X (Y, Z), where X is the metal element and Y and Z are the oxidation states, where Y≠Z. The mixed oxidation state oxide may be one compound, wherein metal ions are in different oxidation states (i.e. X(Y, Z)).

According to some embodiments, the mixed oxidation state oxide useful in the masks of the present invention is selected from the group consisting of tetrasilver tetroxide (TST)—Ag₄O₄ (Ag I, III), Ag₃O₄, Ag₂O₂, tetracopper tetroxide—Cu₄O₄ (Cu I, III), Cu₄O₃, Cu (I, II), Cu (II, III), Co(II, III), Pr(III, IV), Bi(III, V), Fe(II, III), and Mn(II, III) oxides and combinations thereof. Each possibility represents a separate embodiment of the invention. In certain embodiments, the material comprises a mixed oxidation state oxide selected from the group consisting of tetrasilver tetroxide, tetracopper tetroxide and a combination thereof.

As used herein, the term “single oxidation state” refers to atoms, ions or molecules in which same types of atoms are present in one oxidation state only. For example, in copper (I) oxide all copper ions are in the oxidation state +1, in copper (II) oxide all copper ions are in the oxidation state +2 and in zinc oxide all zinc ions are in oxidation state +2.

According to some embodiments, the single oxidation state oxide useful in the masks of the present invention is selected from the group consisting of copper oxide, silver oxide, zinc oxide and combinations thereof.

As used herein, the term “copper oxide” refers to either or both of copper oxide's multiple oxidation states: the first, principal single oxidation state cuprous oxide ((Cu₂O), also identified as copper (I) oxide); or the second, higher single oxidation state cupric oxide ((CuO), also identified as copper (II) oxide) either individually or in varying proportions of the two naturally occurring oxidation states.

As used herein, the term “silver oxide” refers to silver oxide's multiple oxidation states: the first, principal single oxidation state Ag₂O (also identified as silver (I) oxide); or the second, higher single oxidation state AgO, (also identified as silver (II) oxide); or the third highest single oxidation state Ag₂O₃, individually or in any varying proportion of these three naturally occurring oxidation states.

As used herein, the term “zinc oxide” refers to zinc oxide's principal oxidation state ZnO₂.

In certain embodiments, the first fabric layer, the second fabric layer, the third fabric layer, and/or the fourth fabric layer comprise a single oxidation state oxide selected from the group consisting of copper oxide, silver oxide and a combination thereof. In further embodiments, the single oxidation state oxide is copper oxide. In still further embodiments, the first fabric layer, the second fabric layer, the third fabric layer, and/or the fourth fabric layer comprise a single oxidation state oxide selected from the group consisting of Cu₂O, CuO and combinations thereof. In certain embodiments, copper oxide is Cu₂O.

In certain embodiments, the first fabric layer, the second fabric layer, the third fabric layer, and/or the fourth fabric layer comprise a mixed oxidation state oxide selected from the group consisting of tetrasilver tetroxide, Ag₃O₄, Ag₂O₂, tetracopper tetroxide, Cu₄O₃, Cu (I, II), Cu (II, III), Co(II, III), Pr(III, IV), Bi(III, V), Fe(II, III), and Mn(II, III) oxides and combinations thereof.

According to some embodiments, the metal oxides useful in the masks of the present invention are selected from the group consisting of copper oxide, tetracopper tetroxide, silver oxide, tetrasilver tetroxide, zinc oxide and combinations thereof. According to further embodiments, the metal oxides are selected from the group consisting of Cu₂O, CuO, Cu₄O₄, Ag₂O, AgO, Ag₂O₂, Ag₂O₃, Ag₄O₄, ZnO₂ and combinations thereof. In particular embodiments, the first fabric layer, the second fabric layer, the third fabric layer, and/or the fourth fabric layer comprise at least two metal oxides selected from the group consisting of copper oxide, tetrasilver tetroxide, tetracopper tetroxide and combinations thereof. In the currently preferred embodiments, the single oxidation state oxide is copper oxide and the mixed oxidation state oxide is tetrasilver tetroxide. In further embodiments, the single oxidation state oxide is cuprous oxide and the mixed oxidation state oxide is tetrasilver tetroxide.

According to further embodiments, each of the metal oxides can be present in the synergistic combination in a weight percent of from about 0.05% to about 99.95%, such as from about 0.1% to about 99.9%, or from about 0.5% to about 99.5%. Each possibility represents a separate embodiment of the invention. According to some embodiments, the mixed oxidation state oxide constitutes from about 1% to about 20% wt. of the total weight of the combination of the at least two metal oxides. According to further embodiments, the mixed oxidation state oxide constitutes from about 0.05% to about 15% wt. of the total weight of the combination of the two metal oxides, such as from about 0.1% to about 15% wt., from about 0.5% to about 15% wt., from about 1% to about 5% wt., from about 0.5% to about 5% wt., or from about 0.1% to about 3% wt. of the total weight of the combination of the two metal oxides. Each possibility represents a separate embodiment of the invention.

According to particular embodiments, the mixed oxidation state oxide constitutes about 3% wt. of the total weight of the combination of the two metal oxides. According to further particular embodiments, the mixed oxidation state oxide constitutes about 1% wt. of the total weight of the combination of the two metal oxides. According to yet further particular embodiments, the mixed oxidation state oxide constitutes about 0.5% wt. of the total weight of the combination of the two metal oxides. According to still further particular embodiments, the mixed oxidation state oxide constitutes about 0.1% wt. of the total weight of the combination of the two metal oxides. According to yet further particular embodiments, the mixed oxidation state oxide constitutes about 0.05% wt. of the total weight of the combination of the two metal oxides

According to some embodiments, the mixed oxidation state oxide is present in the synergistic combination of the metal oxide powders in a detectable amount. The presence of the mixed oxidation state oxide in the synergistic mixture can be detected by means of an X-ray diffraction spectroscopy (XRD), electron microscopy, electron spectroscopy, Raman spectroscopy or electroanalytical methods. Electron spectroscopy includes, inter alia, X-ray photoelectron spectroscopy (XPS), electron spectroscopy for chemical analysis (ESCA and Auger electron spectroscopy (AES). The non-limiting example of electron microscopy method suitable for the detection of mixed oxidation state oxide is Scanning electron microscopy (SEM), optionally conjugated with Energy-dispersive X-ray spectroscopy (EDS). According to certain embodiments, the presence of the mixed oxidation state oxide is detected by XRD.

According to some embodiments, the particle size of the commercially available metal oxide powder is from about 10 to about 20 microns. The metal oxide powder can be ground to a particle size of from about 1 micron to about 10 microns. Accordingly, the size of the metal oxide particles in the materials of the present invention can be from about 1 nanometer to about 20 microns. According to some embodiments, the particle size is from about 1 to 10 microns. According to further embodiments, the particle size is from about 5 to about 8 microns. According to some embodiments, the metal oxide powders comprise agglomerates which are no larger than 20 microns. According to other embodiments, the metal oxide powders comprise agglomerates which are no larger than 10 microns. In other embodiments, the materials of the present invention are essentially devoid of metal oxide particles agglomerates.

According to some embodiments, the first fabric layer constitutes from about 15% to about 45% of the total weight of the body portion of the mask. According to further embodiments, the first fabric layer constitutes from about 20% to about 40% of the total weight of the body portion of the mask. According to yet further embodiments, the first fabric layer constitutes from about 24.5% to about 35% of the total weight of the body portion of the mask.

According to some embodiments, the second fabric layer constitutes from about 15% to about 45% of the total weight of the body portion of the mask. According to further embodiments, the second fabric layer constitutes from about 20% to about 40% of the total weight of the body portion of the mask. According to yet further embodiments, the second fabric layer constitutes from about 24.5% to about 35% of the total weight of the body portion of the mask.

According to some embodiments, the third fabric layer constitutes from about 15% to about 45% of the total weight of the body portion of the mask. According to further embodiments, the third fabric layer constitutes from about 20% to about 40% of the total weight of the body portion of the mask. According to yet further embodiments, the third fabric layer constitutes from about 24.5% to about 35% of the total weight of the body portion of the mask.

According to some embodiments, the fourth fabric layer constitutes from about 15% to about 45% of the total weight of the body portion of the mask. According to further embodiments, the fourth fabric layer constitutes from about 20% to about 40% of the total weight of the body portion of the mask. According to yet further embodiments, the fourth fabric layer constitutes from about 24.5% to about 35% of the total weight of the body portion of the mask.

As mentioned above, the safety mask according to the principles of the present invention, comprises a nanofiber membrane, which is a middle layer, i.e., being disposed between the first fabric layer and the second fabric layer. The term “nanofiber”, as used herein, refers to fibers having a diameter in the nanometer scale, i.e., of less than about 1000 nm. Preferably, the nanofibers have a diameter of less than about 500 nm. Nanofibrous media have low basis weight, high permeability and small pore size that make them appropriate for a wide range of filtration applications especially for smaller particles. In addition, nanofiber membranes offer unique properties such as high specific surface area (ranging from 1-100 m²/g depending on the diameter of fibers and intrafiber porosity), good interconnectivity of pores and potential to incorporate active chemistry or functionality on a nanoscale. Nanofibers are typically produced onto a substrate, typically a nonwoven fabric. In some embodiments, the nanofiber membrane has random fiber configuration.

Nanometric fibers can be produced, for example, by electro spinning or melt blowing. The nanofibers can be electrospun onto the first fabric layer or the second fabric layer. The nanofibers can be produced from a natural, synthetic or semi-synthetic polymer material, which can be used in electrospinning to obtain the desired nanometric diameter of the spun fiber. Non-limiting examples of suitable polymers include polyvinyl alcohol (PVA), polytetrafluoroethylene (PTFE), polypropylene (PP), polyvinyl chloride (PVC), cellulose, polyethylene, polysulfone, Nylon, polyacrylonitrile, polystyrene, polyethylene oxide (PEO), polyethylene-terephthalate (PET), polyethylene-naphthalate (PEN), and combinations thereof. In certain embodiments, the nanofiber membrane is made of PVA.

The nanofiber membrane can have a thickness of from about 1 μm to about 100 μm. In some embodiments, the nanofiber membrane has a thickness of from about 5 μm to about 50 μm. In certain embodiments, the nanofiber membrane has a thickness of from about 5 μm to about 20 μm.

The nanofiber membrane can have a surface density of from about 0.5 g/m² to about 10 g/m². According to further embodiments, the nanofiber membrane has a surface density of from about 1 g/m² to about 7.5 g/m².

The nanofiber membrane can have a mean pore size of from about 40 nm to about 500 nm. According to some embodiments, the nanofiber membrane has a mean pore size of from about 40 nm to about 300 nm. According to further embodiments, the nanofiber membrane has a mean pore size of from about 40 nm to about 100 nm. According to additional embodiments, the nanofiber membrane has a mean pore size of from about 100 nm to about 400 nm.

The nanofiber membrane can have a porosity of from about 60% to about 95%. In some embodiments, the nanofiber membrane has a porosity of from about 70% to about 90%.

The nanofiber membrane can have a mean fiber diameter of from about 100 nm to about 800 nm. In some embodiments, the nanofiber membrane has a mean fiber diameter of from about 200 nm to about 600 nm.

In some currently preferred embodiments, the nanofiber membrane does not contain the synergistic combination of metal oxides. Incorporation of the metal oxides into the first, the second the third, and the fourth fabric layers, is sufficient in order to impart germicidal properties to the entire body portion of the safety mask, thereby obviating the need to introduce said metal oxides into the nanofiber membrane, in an expensive and complicated process. Additionally, the lack of the metal oxide powders within the nanofiber membrane, increases the air permeability of said membrane and the overall breathability of the safety mask.

According to some embodiments, the nanofiber membrane constitutes from about 2% to about 10% of the total weight of the body portion of the mask. According to further embodiments, the nanofiber membrane constitutes from about 4% to about 8% of the total weight of the body portion of the mask.

According to some embodiments, the metal oxide powders are incorporated into the first, second, third, and/or fourth fabric layers by a master batch manufacturing.

As used herein, the term “master batch” unless otherwise indicated, refers to a carrier polymer containing metal oxide particles, formed into pellets or granules, wherein the polymer is compatible with the end product material. The master batch can be added as a chemical additive to a polymeric slurry comprising same or chemically compatible polymer before extrusion, molding, casting or 3D printing. Alternatively, the master batch can comprise a compounded resin containing the final dosage of the polymers and the metal oxides required for the product to be formed from the polymer.

Metal oxide powders can be included in a polymer using a master batch system so that the powder particles form part of the entire polymeric product. According to some embodiments, the first metal and the second metal of the metal oxide powders, are different. Thus, according to further embodiments, the at least two metal oxide powders have substantially different specific gravities. When two or more particulate compounds having different specific gravities and being disruptive to non-isotactic materials, such as the majority of polymers, have to be incorporated into the polymeric material, control over suspension and dispersion of the particles in the polymeric slurry is complicated. Such slurries generally yield inhomogeneous extruded or cast polymers.

Preferably, the at least two metal oxide powders are incorporated within the fabric layers in a generally uniform fashion. As used herein, the terms “uniform”, “generally uniform”, “substantially uniform” or “homogeneous” that can be used interchangeably, denote, in some embodiments, that the volume percentage of the metal oxide particles within two different 1 cm² sections of the fabric layer varies by less than 20%, preferably less than 10%. In further embodiments, said terms denote that the volume percentage of the particles of the single oxidation state metal oxide within two different 1 cm² sections of the fabric layer varies by less than 20%, preferably less than 10%. In still further embodiments, said terms denote that the volume percentage of the particles of the mixed oxidation state metal oxide within two different 1 cm² sections of the fabric layer varies by less than 20%, preferably less than 10%.

According to some embodiments, the fabric layers of the present invention comprise at least two metal oxide powders having substantially different specific gravities. The term “substantially different specific gravity” refers, in some embodiment, to the variance in the specific gravities of the at least two metal oxide powders, which is higher than about 5%. In another embodiment, the term refers to the variance of higher than about 10%. In yet another embodiment, the term refers to the variance of higher than about 15%.

To accommodate a plurality of metal oxide powders having distinct specific gravities in a single polymeric slurry, it is necessary to compensate for the particle weight differences of the metal oxides. In order to do so, the bulk densities of the metal oxide powders should be equalized. As used herein, the term “bulk density” refers to the mass of many particles of the powder divided by the total volume they occupy. According to some embodiments, the fabric layers comprise at least two metal oxides powders processed to have a substantially similar bulk density. The term “substantially similar bulk density” refers, in some embodiments, to the variance in the bulk density of the at least two metal oxide powders, which is less than about 20%. In another embodiment, the term refers to the variance of less than about 10%. In yet another embodiment, the term refers to the variance of less than about 5%.

For example, specific gravity of copper oxide is 6.0 g/ml, wherein specific gravity of tetrasilver tetroxide is 7.48 g/ml. The bulk densities of the unprocessed copper oxide and the tetrasilver tetroxide powders are thus significantly different. Without wishing to being bound by theory or mechanism of action, in order to be incorporated into the polymer in a substantially uniform manner, the powders have to be processed to equalize the bulk densities thereof. Equalizing the bulk densities of the metal oxide powders can be achieved by altering the particle size of the metal oxide powders. Said particle size alteration can be performed by decreasing or increasing the particle size of the powders. For example, the particles size of the powders can be decreased by grinding and increased by applying a coating. The extent of the increase or decrease in the particle sizes of one metal oxide powder as compared to the other metal oxide powder is dependent on the specific gravities and/or the initial bulk densities of said metal oxide powders.

According to some embodiments, the metal oxide powders are processed by grinding. In other embodiments, the metal oxide powders are processed by milling. According to certain embodiments, the metal oxide powders are processed to have mean particle sizes which are inversely proportional to the specific gravities thereof. According to another embodiment, the metal oxide powders are ground to have a difference in mean particle sizes which is inversely proportional to a difference in the specific gravities thereof. According to the further embodiments, difference in the mean particle sizes of the metal oxide powders is inversely proportional to the difference in the specific gravities thereof.

According to further embodiments, the fabric layers comprise at least two metal oxide powders having essentially similar particle sizes. The term “substantially similar particle size” refers, in another embodiment, to the variance in the particle size of the at least two metal oxide powders which is less than about 20%. In another embodiment, the term refers to the variance of less than about 10%. In yet another embodiment, the term refers to the variance of less than about 5%. In still another embodiment, the term refers to the variance of less than about 1%.

According to further embodiments, the metal oxide powders are processed to have substantially similar particle sizes. According to further embodiments, the metal oxide powders are ground to have substantially similar particle sizes. According to yet further embodiments, at least one of the metal oxide powders is ground to obtain the at least two metal powders having substantially similar particle sizes.

According to some embodiments, the particles of at least one metal oxide powder comprise a coating. According to other embodiments, the particles of at least two metal oxide powders comprise the coating. In some embodiments, at least one of the metal oxide powders is processed to have coated particles. In further embodiments, each of the at least two metal oxide powders is processed to have coated particles. According to certain embodiments, said particles have substantially similar sizes. According to further embodiments, the difference in the coating thickness is proportional to the difference in specific gravity of the metal oxide powders. According to yet further embodiments, the difference in the coating weight is proportional to the difference in specific gravity of the metal oxide powders. According to some embodiments, the at least two metal oxide powders comprise particles having a different coating material. The molecular or specific weight of the coating material can be adjusted to compensate for the difference in the specific gravities of the metal oxide powders.

The metal oxide particles coating may comprise polyester or polyalkene wax. The non-limiting examples of the polyalkene wax include polypropylene wax marketed by Clariant as Licowax PP 230, an oxidized polyethylene wax marketed by Clariant as Licowax PED 521, an oxidized polyethylene wax marketed by Clariant as Licowax PED 121 or an ethylene homopolymer wax marketed by BASF as Luwax®.

According to further embodiments, the coating material comprises a copolymer of polyethylene wax and maleic anhydride. According to yet further embodiments, the coating material further comprises ionomers of low molecular weight waxes. According to additional embodiments, the polyethylene wax has a high wettability. In some embodiments, the coating material comprises homopolymers, oxidized homopolymers, high density oxidized homopolymers and co-polymers of polyethylene, polypropylene and ionomer waxes, micronized polyalkene waxes or mixtures thereof, as well as co-polymers of ethylene-acrylic acid and ethylene-vinyl acetate.

According to some embodiments, the weight of the coating material applied to the powder constitutes from about 0.2% to about 2% wt. of the metal oxide powder weight. According to additional embodiments, the weight of the coating material constitutes from about 0.2% to about 1% wt. of the metal oxide powder weight, preferably from about 0.4 to about 0.5% wt. Each possibility represents a separate embodiment of the invention. In a certain embodiment, the weight of the coating material constitutes about 1% wt. of the metal oxide powder weight.

According to some embodiments, the metal oxides are pretreated with an encapsulating compound. Said compounds isolate the metal oxides so that they will not interact with the polymeric material and are configured to abrade off the powder during product use. The encapsulating compound can be selected from the group consisting of silicates, acrylates, cellulose, protein-based compounds, peptide-based compounds, derivatives and combinations thereof. In some embodiments, the encapsulating compound is selected from the group consisting of silicate, poly(methyl methacrylate) (PMMA) and a combination thereof.

According to some embodiments, the weight of the encapsulating compound applied to the powder constitutes from about 0.2% to about 2% wt. of the metal oxide powder weight. According to additional embodiments, the weight of the encapsulating compound constitutes from about 0.2% to about 1% wt. of the metal oxide powder weight, preferably from about 0.4 to about 0.5% wt. Each possibility represents a separate embodiment of the invention.

According to some embodiments, the fabric layers of the safety mask comprise a metal deactivating agent or a chelating agent associated with the metal oxide powders. As used herein, the terms “metal deactivating agents” and “chelating agents” that can be used interchangeably, refer to an agent generally comprising organic molecules containing heteroatoms or functional groups such as a hydroxyl or carboxyl, the agent acting by chelation of the metal to form inactive or stable complexes. Non-limiting examples of the said metal deactivating agents and/or chelating agents include a phenolic antioxidant, potassium iodide, potassium bromide, calcium stearate, zinc stearate, aluminum stearate, tertiary chain extenders and combinations thereof. According to a particular embodiment, the metal deactivating agent is a phenolic antioxidant. The phenolic antioxidant can be selected from, but not limited to 2′,3-bis [[3-[3,5-di-tert-butyl-4-hydroxyphenyl]propionyl]] propionohydrazide marketed under the name Irganox® MD 1024 by CIBA; Vitamin E (alpha-tocopherol) which is a high molecular weight phenolic antioxidant, marketed under the name Irganox® E 201 by CIBA; Irganox® B 1171, marketed by CIBA, which is a blend of a hindered phenolic antioxidant and a phosphate; and combination thereof. According to certain embodiments, the metal deactivating agents abrade off the metal oxide particles upon hydration of the material.

According to some embodiments, the weight of the metal deactivating agent applied to the powders constitutes from about 0.2% to about 5% wt. of the metal oxide powder weight. According to additional embodiments, the weight of the metal deactivating agent comprises from about 0.5% to about 1% wt. of the metal oxide powder weight. In a certain embodiment, the weight of the metal deactivating agent constitutes about 1% wt. of the metal oxide powder weight.

According to further embodiments, the fabric layers of the safety mask further comprise an additional component, selected from the group consisting of a surfactant, detergent, wetting agent, emulsifier, foaming agent, dispersant and combinations thereof. In some embodiments, said additional component is associated with the metal oxide powder. Non-limiting examples of the surfactant include Sigma Aldrich Niaproof®, Dow Corning Xiameter® and Triton-X-100. The surfactant may further comprise a solvent, such as but not limited to, methyl alcohol, methyl ethyl ketone, or toluene. According to some embodiments, the material is devoid of the surfactant.

According to some embodiments, the weight of the surfactant constitutes from about 0.05% to about 2% wt. of the metal oxide powder weight. In a certain embodiment, the weight of the surfactant constitutes about 0.5% wt. of the metal oxide powder weight.

According to some embodiments, the composition of the master batch, comprising the polymer and the synergistic composition of the metal oxides is formed into a fiber. According to some embodiments, the master batch composition is formed into a fiber by means of extrusion, molding, casting or 3D printing of the polymer, comprising said synergistic combination. The metal oxides can be introduced into the natural material fibers by using, for example, sonochemical cavitation, as described in WO2019/229756 and WO2013/190317, which contents are incorporated by reference herein in their entirety. The obtained fibers can be fabricated into woven or non-woven textiles, as known in the art, and described hereinabove.

In some embodiments, the combined weight of the at least two metal oxides constitutes from about 0.25% to about 50% wt. of the total weight of the first, the second, the third, and/or the fourth fabric layer. In further embodiments, the combined weight of the at least two metal oxides constitutes from about 0.5% to about 30% wt. of the total weight of the first, the second, the third, and/or the fourth fabric layer. In yet further embodiments, the combined weight of the at least two metal oxides constitutes from about 1% to about 15% wt. of the total weight of the first, the second, the third, and/or the fourth fabric layer. In still further embodiments, the combined weight of the at least two metal oxides constitutes from about 1% to about 5% wt. of the total weight of the first, the second, the third, and/or the fourth fabric layer. In yet further embodiments, the combined weight of the at least two metal oxides constitutes from about 0.05% to about 5% wt. of the total weight of the first, the second, the third, and/or the fourth fabric layer.

According to some embodiments, the first fabric layer, the second fabric layer, the third fabric layer, and/or the fourth fabric layer comprise the synergistic combination of the at least two metal oxide powders which are incorporated within the fabric fibers. According to some embodiments, the metal oxides powders are attached to the fabric fibers. According to further embodiments, the powders are attached to the fabric fibers surface. According to other embodiments, the powders are embedded into the fabric fibers. According to further embodiments, the powders are embedded into the fabric fibers' surface. According to other embodiments, the powders are deposited on the fabric surface. According to additional embodiments, the powders are inserted into the fabric fibers. According to further embodiments, the powders are inserted into the fabric surface. According to further embodiments, the metal oxide powders particles protrude from the fabric surface. According to still further embodiments, at least part of the metal oxide powders particles protrudes from the fabric surface. According to some embodiments, at least 10% of the synergistic combination of the metal oxides is present on the surface of the fabric layer. According to further embodiments, at least 5% of the synergistic combination of the metal oxides is present on the surface of the fabric layer. According to still further embodiments, at least 1% of the synergistic combination of the metal oxides is present on the surface of the fabric layer. According to other embodiments, the powders are not exposed on the surface of the fabric layer.

The first fabric layer and the second fabric layer can be identical or different. The third fabric layer and the fourth fabric layer can be identical or different. In some currently preferred embodiments, the first fabric layer is identical to the second fabric layer and the third fabric layer is identical to the fourth fabric layer. In further preferred embodiments, the first fabric layer and the second fabric layer are different than the third fabric layer, and the fourth fabric layer.

The safety mask can be configured for single use or multiple use.

According to some embodiments, the mask is a single-use mask (also termed herein “disposable mask”). In further embodiments, the third and/or the fourth fabric layers of the single-use mask are in a form of a spun bond fabric. In still further embodiments, the third and/or the fourth fabric layers of the single-use mask are made of polypropylene. In some related embodiments, the first and/or the second fabric layers of the single-use mask are in a form of a needle punch fabric comprising polyester and at least one of cotton and viscose.

In some embodiments, the mask is a multiple-use mask (also termed herein “washable mask”). In further embodiments, the third and/or the fourth fabric layers of the multiple-use mask are in a form of a woven fabric. In still further embodiments, the third and/or the fourth fabric layers of the multiple-use mask made of cotton, polyester or any combination thereof. In some related embodiments, the first and/or the second fabric layers of the multiple-use mask are in a form of a needle punch fabric comprising polyester and at least one of cotton and viscose.

In one embodiment, the mask comprises the first and the second fabric layers in a form of the needle punch fabric made of a combination of cotton, viscose and polyester, the third and the fourth fabric layers in a form of a spun bond fabric made of polypropylene, and the middle layer comprising an electrospun PVA nanofiber membrane. In some related embodiments, said mask is a single-use mask.

In one embodiment, the mask comprises the first and the second fabric layers in a form of the needle punch fabric made of a combination of 20% (w/w) cotton, 40% (w/w) viscose, and 40% (w/w) polyester, the third and the fourth fabric layers in a form of a knit fabric made of cotton, and the middle layer comprising an electrospun PVA nanofiber membrane. In further embodiments, the synergistic combination of metal oxides is incorporated into the polyester fibers of the first and second fabric layers. In some related embodiments, said mask is a multiple-use mask.

In one embodiment, the mask comprises the first and the second fabric layers in a form of the needle punch fabric made of a combination of 20% (w/w) cotton, 40% (w/w) viscose, and 40% (w/w) polyester, the third and the fourth fabric layers in a form of a knit fabric made of a combination of 50% (w/w) cotton and 50% (w/w) polyester, and the middle layer comprising an electrospun PVA nanofiber membrane. In further embodiments, the synergistic combination of metal oxides is incorporated into the polyester fibers of the first and second fabric layers. In still further embodiments, the synergistic combination of metal oxides is incorporated into the polyester fibers of the third and fourth fabric layers. In some related embodiments, said mask is a multiple-use mask.

The body portion of the safety mask is typically configured to be placed over a mouth and at least part of a nose of a user such that respiration air is drawn through said body portion.

According to some embodiments, the safety mask has air permeability of at least about 70% air flow, which allows for continued wearing for numerous hours. According to further embodiments, the safety mask has air permeability of at least about 80% air flow or at least about 90% airflow.

According to some embodiments, the third fabric layer constitutes the inner layer of the body portion of the mask, configured to face the mouth and/or the nose of the user. In some embodiments, the fourth fabric layer constitutes the outer layer of the body portion of the mask, configured to face the user's environment.

The body portion of the safety mask can be of a variety of styles and geometries, such as, but not limited to, fold-flat surgical mask, pleated face mask, cone mask, duckbill style mask, trapezoidally shaped mask, and molded cup-shaped mask. The body portion can be configured to isolate the mouth and the nose of the user from the environment. The body portion can be made of inelastic materials. Alternatively, the least some of the fabric layers of the body portion of the safety mask can be composed of elastic materials, allowing for the body portion to be stretched over the nose, mouth, and/or face of the user.

The mask can further include a fastening member, which is connected to the body portion of the mask and configured for attaching the body portion to the user. The fastening member can be a pair of manual tie straps that are wrapped around the head of the user and are connected to one another. Additional types of fastening members can be employed in accordance with various embodiments, as known in the art. Instead of the manual tie straps, for example, the fastening member can include clastic car loops, elastic bands wrapped around the head of the user, a hook and loop type fastener arrangement (e.g., VELCRO® fasteners), or a connection directly attaching the face mask to a hair cap.

Additionally, the configuration of the safety mask can be varied in accordance with the desired face coverage. For example, the safety mask can be made in order to cover both the eyes, hair, nose, throat, and mouth of the user. The safety mask can also incorporate any combination of known face mask features, such as, but not limited to, visors or shields, sealing films, and beard covers.

In some embodiments, the mask is a fold-flat surgical mask. In some embodiments, the mask is a molded cup-shaped mask.

Safety masks are used in a wide variety of applications to protect the human respiratory system from particles suspended in the air, powders and solid or liquid aerosols, which can also include microbes and viruses. Protective masks are generally divided into two categories depending on the purpose of their use: masks which protect the wearer from harmful elements and masks which keep the wearer from spreading harmful elements. Therefore, each type of the mask has its defined purpose, although in some cases more than one protective level can be covered by the same mask. The safety mask according to the principles of the present invention can be used both to protect the wearer from harmful elements, e.g., airborne viruses, and to keep the wearer from spreading said harmful elements. Since both the outer and the inner fabric layers of the mask contain the synergistic antimicrobial combination of metal oxides, viruses and bacteria either from the environment or from user's breath are deactivated by the combination of said layers, even if they pass through the nanofiber membrane filter.

According to some embodiments, the source of viral pathogens is a person in the vicinity of the wearer of the mask and the target of said viral pathogens is the wearer of the mask. According to some embodiments, the source of viral pathogens is the wearer of the mask and the target of said viral pathogens is a person in the vicinity of the wearer of the mask.

According to some embodiments, the method of preventing transmission of viral pathogens comprises wearing the multilayer safety mask of the invention by a user. In certain embodiments, the method comprises positioning the body portion of the mask over a mouth and at least part of a nose of the user such that respiration air is drawn through said body portion.

The safety masks according to the various embodiments of the present invention can be used in combating or inhibiting the activity of microbes or micro-organisms, including, but not limited to, viruses (also termed herein “viral pathogens”), gram-positive bacteria, gram-negative bacteria, fungi, parasites, mold, spores, yeasts, protozoa, algae, and acarii. In some embodiments, the mask is for use in decreasing exposure of the wearer to microbes or micro-organisms. In still further embodiments, the mask is for use in decreasing exposure to microbes or micro-organisms emanating from the wearer. The microbes and micro-organisms can be selected from viruses, gram-positive bacteria, gram-negative bacteria, fungi, parasites, mold, spores, yeasts, protozoa, algac, and acarii. According to some currently preferred embodiments, the virus is an airborne virus. The virus can be selected from the group consisting of a coronavirus, a rhinovirus, an influenza A and/or B virus, a parainfluenza 1, 2 and/or 3 virus, a bocavirus, a human metapneumovirus, an adenovirus, a respiratory syncytial virus (RSV), an enterovirus, varicella-zoster virus, Mycobacterium tuberculosis, measles virus, mumps virus, hantavirus, viral meningitis, and any combination thereof. In some embodiments, the mask is for use in decreasing exposure to a human coronavirus. The human coronavirus can be selected from SARS-COV-2, MERS-COV and SARS-COV. In certain embodiments, the mask is for use in decreasing exposure to SARS-COV-2.

As used herein and in the appended claims the singular forms “a”, “an,” and “the” include plural references unless the content clearly dictates otherwise. Thus, for example, reference to “a fiber” includes a plurality of such fibers and equivalents thereof known to those skilled in the art, and so forth. It should be noted that the term “and” or the term “or” is generally employed in its sense including “and/or” unless the content clearly dictates otherwise.

As used herein, the term “about”, when referring to a measurable value such as an amount, a temporal duration, and the like, is meant to encompass variations of +/−10%, more preferably +/−5%, even more preferably +/−1%, and still more preferably +/−0.1% from the specified value, as such variations are appropriate to perform the disclosed methods.

The following examples are presented for illustrative purposes only and are to be construed as non-limitative to the scope of the invention.

### EXAMPLES

### Example 1

### Metal Oxide Powder Preparation

A tetrasilver tetroxide powder was prepared through a reduction process from a silver nitrate solution by a standard procedure known to a person skilled in the art, and as described by Hammer and Kleinberg in Inorganic Synthesis (volume IV, page 12). It should be further noted that the powder obtained by the described process should be very soft and capable of being converted into a nano-powder with a relative ease.

The basic tetrasilver tetroxide (Ag₄O₄) synthesis as referenced above was prepared by addition of NaOH into distilled water, followed by addition of a potassium persulfate and then the addition of silver nitrate.

The particle size of the powders received varies from nano-particles to agglomerated particles as large as 20 microns. Only powders having a mean particle size of at least 1 micron were used for the preparation of the safety masks to avoid a potentially harmful effect of metal oxide nanoparticles on human health.

The obtained powder was ground down to the desired particle size and mixed with copper oxide. The commercial copper oxide was a cuprous oxide with a purity level of no less than 97% in a 10-20 μm size particle. The powder was ground down to 1 to 5 μm.

### Example 2

### Master Batch Preparation

The metal oxides were incorporated into a polymer using a master batch system so that the powder is embedded on the outside of the polymer and forms part of the entire polymeric product.

To accommodate the different specific gravities of more than one metal oxide in a common master batch, it is necessary to compensate for the differences between the two different metals, should a difference in their weight exist. This is done using two systems as described:

In the first system, the particle sizes of each metal oxide were made equal through proportional size equalization. The specific gravity of copper oxide is approximately 6 g/ml and the specific gravity of tetrasilver tetroxide is 7.48 g/ml. Tetrasilver tetroxide particles were ground down to be approximately 10% to 15% smaller than the copper oxide particles.

In the second system, the particles were all ground to the same size but the heavier particles were coated with a higher amount of polyester wax or polyethylene wax.

The wax was applied in a high sheer mixer in a weight/weight ratio of approximately 10 grams wax to 1000 grams metal oxide. To isolate the metal oxide from a chemical interaction with the carrier polymer, the metal oxide powders were pretreated with an encapsulating compound. The inert encapsulating compounds used were a silicate and Poly(methyl methacrylate) (PMMA). The encapsulation was performed in a high sheer mixer in a weight/weight ratio of approximately 4 g encapsulating agent to 1000 g metal oxide powder.

### Example 3

### Polymer Fiber Preparation

The fabrication of polymeric filament and staple fibers is described hereinbelow. Different amounts of the metal oxide powders can be used based on the desired final concentration in the fibers. The polymer of the fibers was polyester or polypropylenc.

### Filament Fiber

It is noted that the specific gravity of each metal oxide is different and therefore required a treatment of a different coating compound or applying different amount of the same coating compound so that both metal oxide powders would be homogeneously dispersed in the liquid polyester slurry. The metal oxide particles were mixed with the carrier and formed into pellets. As it relates to filament fiber this produced a total of 50 kilo of master batch which is a total of the copper oxide and the tetrasilver tetroxide together. The proportion of the carrier to active material was 5:1 yielding a 20% wt. concentration of the metal oxides in the master batch. 50 kilos of the master batch were mixed into an extrusion tank for spinning through a spinneret and were sufficient to produce 1 ton of a filament polymeric fiber yielding a total of about 1% wt. final concentration of the two metal oxides together in the polymeric fiber. The loading of the metal oxides in a filament fiber can be as high as 4% wt.

### Staple Fiber With Protruding Metal Oxide Powders

For the production of a staple fiber, 28.5 kilo of copper oxide having particle size ground to 1 to 5 microns and 1.5 kilo of tetrasilver tetroxide ground to 1 to 5 microns were mixed with 120 kilos of the chosen carrier polyester polymer for the creation of a master batch. The specific gravity of each compound was different and therefore required a coating by a different coating compound, such as Clariant Licowax PP230 and BASF Luwax® or by different amounts of said compounds, such that the metal oxide particles would be homogeneously dispersed in the suspension. The compounds were mixed with the carrier and were formed into pellets. This produced a total of 150 kilo of master batch. The 150 kilo of master batch was mixed into an extrusion tank for spinning through a spinneret and was sufficient to produce 1 ton of a polymeric staple yarn yielding a total of a about 3% wt. final concentration of the two compounds in the polymer fiber.

### Staple Fiber With Enclosed Metal Oxide Powders

A polyester staple fiber was prepared by combining copper oxide powder which constituted 2.85% wt. of the total weight of the fiber and tetrasilver tetroxide powder which constituted 0.015% wt. of the total weight of the fiber. The particle size of the metal oxides was brought down to between 0.25 to 0.35 microns and the powders were incorporated directly into the polymer fiber. The process included milling the powders to the desired size, placing the powders on the fiber and passing the fiber with the powders through a trough of water though which ultrasonic waves were passed.

### Example 4

### Cotton Fiber Preparation

Metal oxides were introduced into cotton fibers using sonochemical cavitation process, as described, inter alia, in WO2019/229756. In brief, cotton sliver fibers were placed and conveyed through a designated system. The fibers were conveyed through a wetting bath containing a deacrating agent. After squeezing out excess water, the damp sliver fibers were conveyed to a paste dispenser where both sides of the sliver ribbon were coated with paste. The paste contained copper oxide/TST/water/thickening agent. The paste was dispensed on the moist sliver ribbon.

The paste-coated sliver ribbon was advanced to a bore sonotrode and passed through a bore of the sonotrode where the coated sliver ribbon was subjected to ultrasonic waves. The sliver ribbon was then brought to a washing system where it underwent a hot water shower.

### Example 5

### Woven Fabric Preparation

The system for preparation of woven fabrics from staple fibers follows the standard method as is common in the industry. After the staple fibers were prepared and were in bale form, they were put through a carder. The carder is a large cylinder with teeth which straightens out the fibers and makes them parallel to one another. The parallel fibers were formed into a very light web. The web was then twisted to form a tow. The tow was then more tightly spun to form the yarns. The thickness of the yarns is a function of how tightly the yarns were pulled and twisted. The yarns were then woven as is standard to the industry.

The particular woven fabrics prepared in accordance with the described procedure, which were used in the safety masks of the present invention and which biocidal efficiency was tested as shown in Example 9 hereinbelow, included:

Knit 100% cotton fabric which contained 3% (w/w) CuO and 0.1% (w/w) TST; and

Woven 50% (w/w) cotton-50% (w/w) polyester, wherein the polyester fibers contained 3% (w/w) CuO and 0.1% TST woven fabric.

### Example 6

### Non-Woven Fabric Preparation

Fibers in a non-woven fabric were extruded in a row with a few thousand spinneret holes through which the slurry was run. The slurry solidified upon exposure to the air but immediately after the extrusion the newly formed fibers were exposed to high pressure streams of air which cause the fibers to intermingle forming a sheath. The weight of the fabric is a function of the speed of the extrusion. The faster the extrusion, the lighter the fabric.

### Spun Bond Fabric Preparation

A master batch pellet was formulated so that it contained between 20% and 40% (w/w) of the synergistic combination of metal oxides to the weight of the polymer. This master batch was then added to the polypropylene or polyester when it was in liquid form before extrusion. The desired dosage is calculated (usually around 3% of the synergistic combination (w/w) to slurry), mixed, and extruded.

### Needle Punch Fabric Preparation

A master batch pellet was formulated so that it contained between 20% and 40% of the synergistic combination of metal oxides (w/w) to the weight of the polymer. This master batch was then added to the polypropylene or polyester when it was in liquid form before extrusion. The desired dosage was calculated (usually around 3% of the synergistic combination (w/w) to slurry), mixed, and extruded.

The obtained fibers were chopped and formed into a staple fiber with a denier between 1 and 6 in thickness. These fibers were then mixed in a card with other fibers which were usually a combination of cotton and viscose. The carded fibers were then converted to a thin layer which was usually 3 to 4 meters wide (depending on the machinery). These layers were then placed one on top of the other so that each layer is at a 90-degree angle to the one below it. These layers, usually 6 to 8 layers in number, were then intermingled by use of a series of needles which have a barb on them so that when the needles rise and fall, they cause an intermingling of the fibers. As is usual in the industry, the layers were then put through a heat press to compress them thus forming the needle punch layer.

### Example 7

### Multiple Use Safety Mask Fabrication

### Multiple Use Mask Comprising Inner and Outer Cotton Layers

Two non-woven (needle punch) fabric layers made of 40% (w/w) polyester, 20% (w/w) cotton and 40% (w/w) viscose and two woven (knit) fabric layers comprising cotton were prepared as described in Examples 5-6. The synergistic combination of metal oxides was present in the polyester layer of the needle punch fabric and in 10% of the knit cotton fabric.

Electrospun PVA nanofiber membrane was obtained from Respilon.

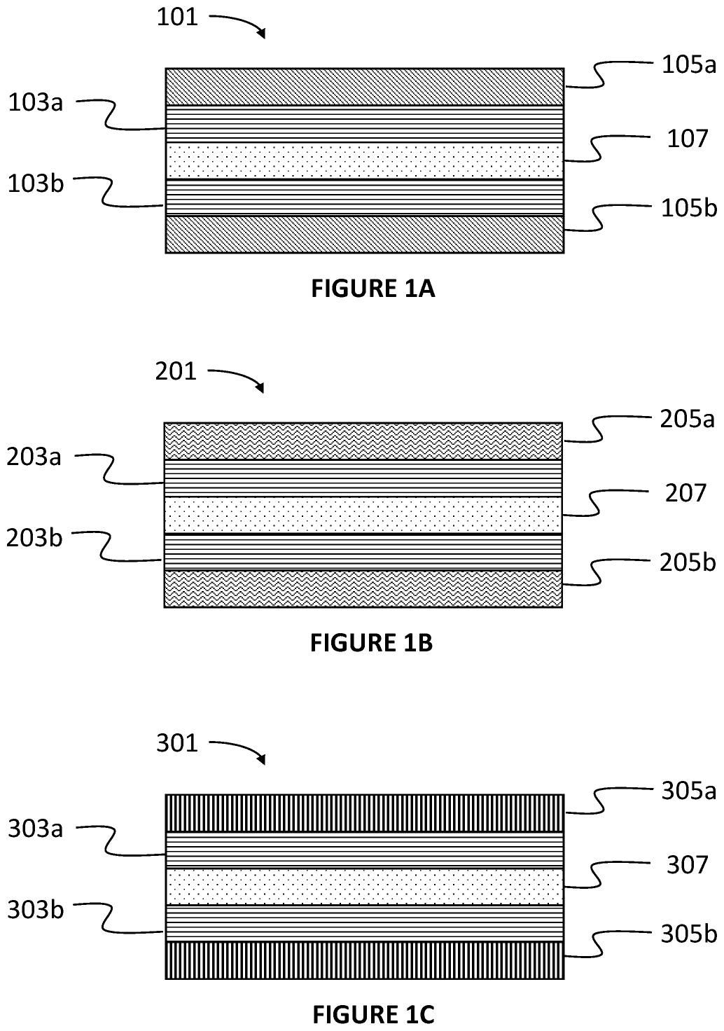

The multiple use safety mask was assembled as follows (FIG. 1A):

The multiple use safety mask 101 is made up of 5 layers—first layer 103a, which is a needle punch fabric, second layer 103b, which is a needle punch fabric, third layer 105a, which is a cotton knit fabric, fourth layer 105b, which is a cotton knit fabric, and PVA nanofiber membrane 107. The third fabric layer 105a and the fourth fabric layer 105b are the inner layer and outer layers of the safety mask 101. The first fabric layer 103a is disposed between the third fabric layer 105a and the nanofiber membrane 107 and the second fabric layer 103b is disposed between the fourth fabric layer 105b and the nanofiber membrane 107.

### Multiple Use Mask Comprising Inner and Outer Cotton/Polyester Layers

Two non-woven (needle punch) fabric layers made of 40% (w/w) polyester, 20% (w/w) cotton and 40% (w/w) viscose and two woven fabric layers comprising 50% (w/w) cotton/50% (w/w) polyester were prepared as described in Examples 3-6. The synergistic combination of metal oxides was present in the polyester fibers of the needle punch fabric and in up to 40% of the weft of the woven fabric, being polyester.

Electrospun PVA nanofiber membrane was obtained from Respilon.

The multiple use safety mask was assembled as follows (FIG. 1B):

The multiple use safety mask 201 is made up of 5 layers—first layer 203a, which is a needle punch fabric, second layer 203b, which is a needle punch fabric, third layer 205a, which is a cotton/polyester woven fabric, fourth layer 205b, which is a cotton/polyester woven fabric, and PVA nanofiber membrane 207. The third fabric layer 205a and the fourth fabric layer 205b are the inner layer and outer layers of the safety mask 201. The first fabric layer 203a is disposed between the third fabric layer 205a and the nanofiber membrane 207 and the second fabric layer 203b is disposed between the fourth fabric layer 205b and the nanofiber membrane 207.

### Example 8

### Single Use Safety Mask Fabrication

Two non-woven (needle punch) fabric layers made of 40% (w/w) polyester, 20% (w/w) cotton and 40% (w/w) viscose and two spun bond fabric layers comprising polypropylene were prepared as described in Examples 3-6. The synergistic combination of metal oxides was present in the polyester fibers of the needle punch fabric and in polypropylene fibers of the spun bond fabric.

Electrospun PVA nanofiber membrane was obtained from Respilon.

The single use safety mask was assembled as follows (FIG. 1C):

The single use safety mask 301 is made up of 5 layers—first layer 303a, which is a needle punch fabric, second layer 303b, which is a needle punch fabric, third layer 305a, which is a polypropylene spun bond fabric, fourth layer 305b, which is a polypropylene spun bond fabric, and PVA nanofiber membrane 307. The third fabric layer 305a and the fourth fabric layer 305b are the inner layer and outer layers of the safety mask 301. The first fabric layer 303a is disposed between the third fabric layer 305a and the nanofiber membrane 307 and the second fabric layer 303b is disposed between the fourth fabric layer 305b and the nanofiber membrane 307.

### Example 9

### Antimicrobial Properties of the Safety Mask Fabric Layers

To determine the virucidal effectiveness of the safety mask against human coronavirus, each fabric layer of the mask was tested according to modified ASTM E1053. The tested samples included five plastic bags, each contained one textile swatch of each fabric material. Detailed description of the samples' composition is summarized in Table 1. For absorbency test, circular swatches (2 cm2) of each fabric material were placed in sterile petri dishes, 100 μl plain medium (MEM with 5% fetal bovine serum (FBS) was added on the center of each swatch to observe if the inoculum was completely absorbed on the fabric and held by it. In case the swatches failed to hold the required amount of inoculum, more than one swatch was used, as was the case for DP3 and DP4, for which two swatches were used and DP5, for which three swatches were used. Then 100 μl of the human corona virus 229E, ATCC VR-749 or plain media inoculated the center of each swatch for 5-minute contact time at room temperature. The absorbency test results demonstrated poor absorbency for DPI and DP2 samples, thus the virus or media in these swatches were gently vortexed.

Each swatch was further neutralized with 1 ml neutralizer medium (MEM supplemented with 5% FBS), mixed by pipetting and transferred to a sterile tube from which a serial 10-fold dilutions were prepared with the dilution medium (MEM with 5% FBS). For In vitro infectivity assay of the recovered virus, host cells (MRC-5 ATCC CCL-171) grown for 28 days in MEM WITH 10% FBS 1×P/S were plated in 96 well plates at seeding density of 2×105 cells/ml and incubated 1 day prior to the test at 37±1° C., 5% CO2 and 95% RH. After incubation, the host cells were inoculated with 100 μl/well of tests and controls in quadruplicate.

TABLE 1
Composition and virucidal effectiveness of the samples

| Sample # | DP1 | DP2 | DP3 | DP4 | DP5 |
|---|---|---|---|---|---|
| Layer # in | 3 and 4 | 3 and 4 | 1 and 2 | 3 and 4 | 3 and 4 |
| the mask | | | | | |
| Type of | 100% | polypropylene | 40% polyester/ | 50% | polypropylene |
| material* | cotton | | 40% viscose/ | polyester/ | |
| | | | 20% | 50% | |
| | | | cotton | cotton | |
| Material into | cotton | polypropylene | polyester | polyester | polypropylene |
| which metal oxides | | | | | |
| are incorporated | | | | | |
| Type of fabric | knit | spun bond | needle punch | woven | spun bond |
| | sleeve | 13.5 gram | non-woven | | 30 gram |
| Weight percentage | 3% | 3% | 3% | 3% | 3% |
| of each metal oxide | CuO + | CuO + | CuO + | CuO + | CuO + |
| within the treated | 0.1% | 0.1% | 0.1% | 0.1% | 0.1% |
| fibers (%(w/w)) | TST | TST | TST | TST | TST |
| Weight percentage | 0.3% | 3% | 1.2% | 0.3% | 3% |
| of each metal oxide | CuO + | CuO + | CuO + | CuO + | CuO + |
| within the fabric | 0.01% | 0.1% | 0.04% | 0.01% | 0.1% |
| layer (%(w/w)) | TST | TST | TST | TST | TST |
| Fibers in which | 10% of | filament | staple | 20% of | filament |
| the metal oxide | overall | | | the | |
| are present | knit | | | weft | |
| | fabric | | | only | |
| Metal oxides | 1 to 1.5 | 1 to 1.5 | 1 to 1.5 | 1 to 1.5 | 1 to 1.5 |
| particle size | microns | microns | microns | microns | microns |
| Log10TCID50 | ≤1.5 | 3.0 | 2.85 | 1.85 | 3.0 |
| (Average) | | | | | |
| Covid19 | | | | | |
| Log Reduction | ≥3 | 1.5 | 1.65 | 2.65 | 1.5 |
| (Average) | | | | | |
| Covid19 | | | | | |

*if the fabric is made of a combination of materials, indicated percentages are weight percentages of a material within the total weight of the combination of materials (%(w/w)).

Presence or absence of viral infection was monitored and recorded based on the viral cytopathic effect (CPE) on the host cell, which were distinguishable from the cytotoxic effect induced by the test article.

The controls examined in the virucidal effectiveness included

Cell viability control—which demonstrates that the host cells remain viable throughout the assay. Four wells received culture media only.

Virus control—Determines the positive control virus titer that recovered from a plate at the same time of test sample. Recovered virus titer must be at least four logs.

Cytotoxicity control—Determines extent of cytotoxicity, if any. Serial dilutions were prepared similarly to the test procedure but instead of inoculation with the virus, a cell culture media is used. 10-fold dilutions were prepared and four wells per dilution were inoculated.

Neutralization control—Serial dilutions were prepared similarly to the test procedure but instead of inoculation with the virus, a cell culture media is used. The diluted samples were mixed with approximately 100 TCID50 of test virus and four wells per dilution were inoculated. The samples were incubated under the same conditions as the test sample.

Virus control met the requirements with minimum 104 TCID50 recovered. Cell viability control met the requirements and no contamination occurred. Neutralization control demonstrated that there was no carry-over virucide in the dilutions relied upon to assess whether or not the virus survived the treatment with the disinfectant.

The results of the virucidal screening test are summarized at TABLE 1. The samples tested demonstrated between 1.2 and 2.8 log10 reduction of the virus. Lot DPI showed ≥3 log virus reduction. It can therefore be concluded that one fabric layer of the mask has between 95% and 99.9% virus deactivation rate.

### Example 10

### Viral Filtration Efficiency (VFE) of the Nanofiber Membrane

To determine the filtration efficiency of the nanofiber membrane against viral infection, a VFE test was performed according to ASTM F2101. A suspension of bacteriophage ΦX174 was aerosolized using a nebulizer and delivered to a ˜40 cm² nanofiber membrane at a constant flow rate of 28.3 L/min and fixed air pressure at 21±5° C. and 85±5% RH for a minimum of 4 hours. The challenge delivery was maintained at 1.1−3.3×103 plaque forming units (PFU) with a mean particle size (MPS) of 3.0±0.3 μm. The aerosol droplets were drawn through a six-stage, viable particle, Andersen sampler for collection.

The viral control counts upstream of the sample area were compared to the counts downstream and the viral filtration efficiency percentages (% VFE) were calculated via equation I:

$$\% V F E = \frac{C - T}{C} \times 100 , \qquad Equation I$$

Where:

C is the positive control average calculated to be 2.5×10³ PFU

T is the plate count total recovered downstream of the sample area % VFE demonstrated in Table 2 indicates a very high filtration efficiency of the nanofiber membrane against the viral infection.

TABLE 2
% VFE of nanofiber membrane samples

| Sample number | Percent VFE (%) |
|---|---|
| 1 | >99.9 |
| 2 | 99.8 |
| 3 | 99.7 |
| 4 | >99.9^{a} |
| 5 | >99.9^{a} |

^{a}There were no detected plaques on any of the Andersen sampler plates for this test article.

### Example 11

### Antiviral Activity of the Safety Mask Against H1N1 Virus

The single use safety mask of Example 8 and standard cloth set as control were inoculated with Influenza A virus (H1N1) at a concentration of 2.0×10⁷ (PFU/ml). The common logarithm average of infectivity titer values of both test samples presented in Table 3, demonstrate a slight reduction 2 h after the standard cloth was inoculated. Even lower infectivity titer value was found for the safety mask, indicating a better antiviral activity. The antiviral activity value was further calculated as can be seen in Table 3.

Verifications for the antiviral test showed that the safety mask has no cytotoxic effect

TABLE 3
Antiviral activity of the safety mask against H1N1 virus

| | | The common logarithm | The antiviral |
|---|---|---|---|
| | | average of infectivity | activity value |
| Test sample | | titer value | Ig(Va) − Ig(Vc) |
| Standard | Immediately | Ig(Va) 6.76 | — |
| cloth | after | | |
| | inoculation | | |
| | After 2 h | Ig(Vb) 6.05 | — |

| | | |
|---|---|---|
| Safety mask (Before wash) | Ig(Vc) 2.30 | 4.5 |

### Example 12

### Experimental Methods

### Detection of the Mixed Oxidation State Oxide in the Polymer Material

A portion of textiles or fibers is put in an oven and brought to a temperature which allows the polymer to be carbonized to dust, but which is below the melting temperature of the metal oxides. The dust is then placed in an X-Ray Diffraction system which identifies crystalline structure of a crystal and as such can detect the presence of the metal oxides powders in the sample, which are present in addition to the carbon dust.

### Measurement of the Fabric Mean Pore Size

As the fabrics are created, whether woven, knit, or non-woven, there is a natural space that forms between the fibers. The space will vary from 0.5 microns to 20 microns depending on the fiber or yarn size and how thick the sheath is. In order to reduce the pore size, the fabrics are layered to reduce the average pore size as one fabric blocks the pores of the other. The ultimate size of the visible holes is measured through the passage of a light through the fabric. The more layers there are in the fabric, the smaller are the pores on the top surface.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1-38. (canceled)

39. A multilayer safety mask having antimicrobial properties, said multilayer safety mask comprising:
a body portion including:
at least a first fabric layer and a second fabric layer having random fiber configuration;
a middle layer including a nanofiber membrane; and
a third and a fourth fabric layers,
wherein the first fabric layer is disposed between the third fabric layer and the nanofiber membrane and wherein the second fabric layer is disposed between the fourth fabric layer and the nanofiber membrane; and
wherein the first, the second, the third, and the fourth fabric layers have incorporated therein a synergistic combination of at least two metal oxide powders, including a mixed oxidation state oxide of a first metal and a single oxidation state oxide of a second metal, said combination having synergistic antimicrobial properties, wherein the ions of the metal oxides are in ionic contact upon exposure of said fabric layers to moisture.

40. The multilayer safety mask according to claim 39, wherein the first and the second fabric layers are in a form of a needle punch fabric.

41. The multilayer safety mask according to claim 39, wherein the third and the fourth fabric layers are in a form of a non-woven fabric, selected from the group consisting of a spun bond fabric, melt blown fabric, and combinations thereof.

42. The multilayer safety mask according to claim 39, wherein the first, the second, the third, and the fourth fabric layers have a thickness of from about 100 μm to about 1000 μm; wherein the first, the second, the third, and the fourth fabric layers have a surface density of from about 5 g/m² to about 70 g/m²; wherein the first, the second, the third, and the fourth fabric layers have a mean pore size of at least about 30 μm.

43. The multilayer safety mask according to claim 39, wherein the third and the fourth fabric layers are in a form of a woven or knit fabric; wherein the woven or knit fabric has a surface density of from about 5 g/m² to about 70 g/m² and a mean pore size of from about 20 μm to about 60 μm.

44. The multilayer safety mask according to claim 39, wherein the first, the second, the third, and the fourth fabric layers are made of a synthetic material, semi-synthetic material, natural material, or any combination thereof; wherein the synthetic material or semi-synthetic material includes a polymer selected from the group consisting of polyalkene, polyester, polyamide, polyaramid, cellulose-based polymer, and combinations thereof; and, wherein the natural material is selected from the group consisting of cotton, silk, wool, linen, and combinations thereof.

45. The multilayer safety mask according to claim 39, wherein the combined weight of the at least two metal oxide powders within the fabric layers constitutes from about 0.05% wt. to about 5% wt. of the total weight of each one of the first, the second, the third, and the fourth fabric layers; and wherein the mixed oxidation state oxide is selected from the group consisting of tetrasilver tetroxide (Ag₄O₄), Ag₃O₄, Ag₂O₂, tetracopper tetroxide (Cu₄O₄), Cu (I, III) oxide, Cu (II, III) oxide, and combinations thereof.

46. The multilayer safety mask according to claim 39, wherein the single oxidation state oxide is selected from the group consisting of copper oxide, silver oxide, zinc oxide, and combinations thereof.

47. The multilayer safety mask according to claim 39, wherein the synergistic combination of at least two metal oxide powders includes copper oxide and tetrasilver tetroxide.

48. The multilayer safety mask according to claim 39, wherein the mixed oxidation state oxide constitutes up to about 15% wt. of the total weight of the synergistic combination of at least two metal oxide powders and wherein the mixed oxidation state oxide is present in the synergistic combination in a detectable amount.

49. The multilayer safety mask according to claim 39, wherein each one of the first, the second, the third, and the fourth fabric layers constitutes between about 15% and 45% of the total weight of the body portion of the mask.

50. The multilayer safety mask according to claim 39, wherein the nanofiber membrane is electrospun or melt blown; wherein the nanofiber membrane has a thickness of from about 1 μm to about 100 μm.

51. The multilayer safety mask according to claim 50, wherein the nanofiber membrane has a thickness of from about 5 μm to about 20 μm.

52. The multilayer safety mask according to claim 39, wherein the nanofiber membrane has a surface density of from about 0.5 g/m² to about 10 g/m²; wherein the nanofiber membrane has a mean pore size of from about 40 nm to about 100 nm; wherein the nanofiber membrane has a porosity of from about 70% to about 90%; and wherein the nanofiber membrane has a mean fiber diameter of from about 100 to about 800 nm.

53. The multilayer safety mask according to claim 39, wherein the nanofiber membrane is made of a natural, synthetic or semi-synthetic polymer material selected from the group consisting of polyvinyl alcohol (PVA), polyvinyl chloride (PVC), cellulose, polyethylene, polysulfone, Nylon, polyacrylonitrile, polystyrene, polyethylene oxide (PEO), polyethylene-terephthalate (PET), polyethylene-naphthalate (PEN), and combinations thereof; and wherein the nanofiber membrane constitutes between about 2% and 10% of the total weight of the body portion of the mask.

54. The multilayer safety mask according to claim 39, which is a single-use mask, wherein the third and the fourth fabric layers are in a form of a spun bond fabric, made of polypropylene.

55. The multilayer safety mask according to claim 39, which is a multiple-use mask, wherein the third and the fourth fabric layers are in a form of a woven fabric, made of cotton, polyester or any combination thereof.

56. The multilayer safety mask according to claim 39, wherein the first and the second fabric layers are in a form of a needle punch fabric, which is made of a combination of polyester, cotton, and viscose.

57. The multilayer safety mask according to claim 56, wherein the synergistic combination of at least two metal oxide powders is incorporated into polyester.

58. The multilayer safety mask according to claim 39, for use in combating or inhibiting the activity of microbes or micro-organisms, selected from the group consisting of viruses, gram-positive bacteria, gram-negative bacteria, and any combination thereof; wherein the virus is selected from the group consisting of a coronavirus, a rhinovirus, an influenza A and/or B virus, a parainfluenza 1, 2 and/or 3 virus, and any combination thereof.
